(19) United States
(12) Patent Application Publication
(10) Pub. No.: US 2022/0273177 A1
(43) Pub. Date: Sep. 1, 2022

(54) COVID Thermal Wristband

(71) Applicants: Elmer TOLENTINO, CORONA, CA (US); Juan Vera, Corona, CA (US)

(72) Inventors: Elmer TOLENTINO, CORONA, CA (US); Juan Vera, Corona, CA (US)

(21) Appl. No.: 17/187,715

(22) Filed: Feb. 26, 2021

(51) Int. Cl. A61B 5/01; A41D 13/11; A44C 5/00; A61B 5/00; A61B 90/98; G01K 11/12

(52) U.S. Cl. CPC A61B 5/01; A41D 13/11; A44C 5/0053; A61B 5/742; A61B 5/681; A61B 90/98; A61B 5/6803; G01K 11/12; A61B 2562/0276

(57) ABSTRACT

Improvements in a COVID thermal wristband or facemask that uses a color changing material so the temperature of the wearer can be seen on the user where the wristband or mask contacts the skin of a wearer. The homogeneous nature of the material allows the wristband to be used continuously in dry and wet conditions and is not affected by deep emersion or continuous emersion in water and by chemicals or cleaning solvents in the water or on the hands of the wearer. The wristband can have a one-way ratcheting strap where a user can simply install the band at a loose setting and pull on the strap end to tighten the strap. An RFID data device can be included for contact tracing. A person, parent or teacher can simply look at a wristband to see if there is an elevated temperature.

### CROSS REFERENCE TO RELATED APPLICATION

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

This invention relates to improvements in detecting a skin temperature in a person. More particularly, the present COVID thermal wristband can be continuously warn by a person and does not require any electricity to provide notification that the person may have COVID or a temperature.

### Description of Related Art Including Information Disclosed Under 37 CFR 1.97 and 1.98

In 2019 a pandemic effected the world. A virus designated COVID 19 caused illness to people. Diagnostic of the virus was determined by a nasal swab. One symptom of infection was an elevated temperature that was detected by a thermal scan. Detection of an elevated temperature could be taken as a non-invasive test but required specialized equipment and a power source. Some crystal material change color when subjected to a temperature. These are often formed in a strip, placed on the skin of a person and the temperature of the person on the on the strip is shown. Some jewelry, that was once called “mood rings” or other jewelry changes color based upon the contact temperature of the wearer.

A number of patents and or publications have been made to address these issues. Exemplary examples of patents and or publication that try to address this/these problem(s) are identified and discussed below.

U.S. Pat. No. 9,974,441 issued on May 22, 2018 to Seth O. Lartey et al and is titled Fever Detector Wrist Band. This patent discloses a fever detecting wrist band device comprises a flexible band and a fever detection device attached to the flexible band. The fever detection device comprises a thermochromic liquid crystal (TLC) material, which can change colors corresponding to a set of temperature ranges, encased in a transparent casing. The transparent casing has a thin bottom section to allow the TLC material to gather body heat when the wrist band is worn by a user and the bottom section comes in contact with the skin of the user. While this patent has a color changing area the color change is in a single button and not the entire band.

U.S. Pat. No. 10,565,489 issued on Feb. 18, 2020 to Luke Christopher Waidmann et al., and is titled Spoolable small-form-factor RFID-enabled wristband. This patent discloses a spoolable RFID-enabled wristbands with maximized read range. In an embodiment, a wristband comprises flexible material formed into a flag portion and a strap portion. The flag portion comprises a radio-frequency identification (RFID) inlay embedded within the material. While this patent has an RFID, it does not provide an indicator that the wearer has a temperature.

U.S. Pat. No. 3,802,945 issued on Apr. 9, 1974 to Bill G. James and is titled Heat Sensitive Novelty Device. This patent discloses a novelty device which utilizes the iridescent qualities of liquid crystalline material to effect variations in colorations of the device upon application of different temperatures. This novelty item changes color based upon a temperature it is not made as a homogeneous band that identifies when a person has a temperature.

U.S. Patent Publication Number 2006/0099353 was published on May 11, 2006 to Yi-Ching Needham and is titled Abstract Color Changing Pattern Configuration. This publication discloses an abstract color change pattern configuration, which is primarily structured to comprise a transitional layer and a protective layer. The transitional layer is fabricated from a plurality of temperature variant microcapsule liquid crystal materials, wherewith a plurality of mixing layers of color change microcapsule liquid crystal material are able to transform into irregular patterns, and blended colors manifested therefrom are not limited to generating one pattern, but color blending effectuates a plurality of transformations. Such a configuration realizes fabrication of the transitional layer to produce diverse temperature variant transitional abstract patterns according to user requirements. While the publication discloses color changing it is not a wristband and does not identify when the wearer is sick.

What is needed is a COVID thermal wristband that wraps around a wrist of a user and provides a continuous indication on the thermal health of the user.

### BRIEF SUMMARY OF THE INVENTION

It is an object of the COVID thermal wristband to be made from color changing material. The entire wristband is made from the color changing material so the temperature of the wearer can be seen from all around the wrist of the user. The homogeneous nature of the material allows the wristband to be used continuously in dry and wet conditions and is not affected by deep emersion or continuous emersion in water and by chemicals or cleaning solvents in the water or on the hands of the wearer.

It is an object of the COVID thermal wristband to be made from a thermochromic pigment with a universal copolymer. The color of the universal copolymer becomes less as the temperature increases until the plastic thermochromic pigment becomes completely colorless or white. When the temperature of the wristband decreases the color becomes stronger and returns to its original start color. This type of thermochromic plastic is referred to as a reversible plastic since it will repeat the noted color change an unlimited number of times.

It is an object of the COVID thermal wristband to be made from a flexible material that can be stretched around and/or over the hand of the user. This allows the band to be secured around the wrist of a user like a wristwatch or simply stretched over the hand like a rubber band. Contact with the skin of the user makes conduction of the temperature of the user to the band. The a thermochromic pigment material in the band will change the color of the band from gray to an orange or other color based upon the thermochromic pigment material.

It is another object of the COVID thermal wristband to include a ratcheting strap where a user can simply install the band at a loose setting and pull on the loose end to tighten the strap around the wrist of the user. In an ideal size of the wristband the entire interior area of the wristband is in conductive contact with the skin of the wearer. The ratcheting is with one or more one-way teeth. The wristband can be loosened by “looping” the band over the one-way teeth.

It is another object of the COVID thermal wristband to have an RFID data device. An RFID device does not require internal power for communication and can be used as identification to identify a wearer or to tract movement of the user for contact tracing by logging movement of the RFID tag. The RFID tag can provide anonymous tracking of a person until a band is identified as having an elevated temperature. The tracking database can then be queried to identify others that have been in the same general area. The database can identify a number, user ID, student ID or the name of people.

It is still another object of the COVID thermal wristband to be useful for school children or others for non-invasive temperature detection. A parent or teacher can simply look at wristbands as children enter, leave, play or ask all of the children to lift their arm to view the color of the wristbands as a collective group or in a classroom to identify any individual or individuals that exhibit an elevated temperature. The color change is typically gray or orange with minimal transition color so there is no subjectivity to a temperature change provided there is conductive heat transfer by skin contact.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows a perspective view of a COVID thermal wristband.

FIG. 2 shows a detailed view of the COVID thermal wristband on a wrist of a person.

FIG. 3 shows the securing mechanism on the COVID thermal wristband with the mechanism displaced.

FIG. 4 shows the securing mechanism on the COVID thermal wristband with the mechanism secured.

FIG. 5 shows a cross-section view of the COVID thermal wristband.

FIG. 6 shows a perspective view of an alternate embodiment of the COVID thermal wristband.

FIG. 7 shows another view of the COVID thermal wristband showing an elevated temperature.

FIG. 8 shows the thermal material on a facemask.

FIG. 9 shows a graph of the color change difference.

### DETAILED DESCRIPTION OF THE INVENTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, but is merely representative of various embodiments of the invention. The illustrated embodiments of the invention will be best understood by reference to the drawings, wherein like parts are designated by like numerals throughout.

### ITEM NUMBERS AND DESCRIPTION

11 alternate embodiment wristband
18 head
19 wrist or forearm
20 COVID thermal wristband
21 facemask
30 front bridge
31 rear bridge
40 front tab
41 rear tab
50 bridge opening
51 rear bridge opening
60 strap front body
61 strap buckle base
62 strap tail
63 tail body
64 loop
70 strap holes
80 emblem
81 color change
82 default (cold) color
83 proud
84 ear strap
89 RFID
90 wristband
91 indicia
92 default (cold) color
93 warm color
95 clasp(s)
96 stretch
97 slack
98 pull
99 install
100 color change graph
101 color difference
102 temperature
110 plot
111 cold color
112 color change
114 warm color
120 lower inflection point
121 upper inflection point

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated. The terminology used herein is for the purpose of describing particular embodiments only and is not intended to be limiting of the technology. As used herein, the singular forms “a,” “an,” and “the” are intended to include the plural forms as well, unless the context clearly indicates otherwise.

It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof. It will be understood that like or analogous elements and/or components, referred to herein, may be identified throughout the drawings with like reference characters.

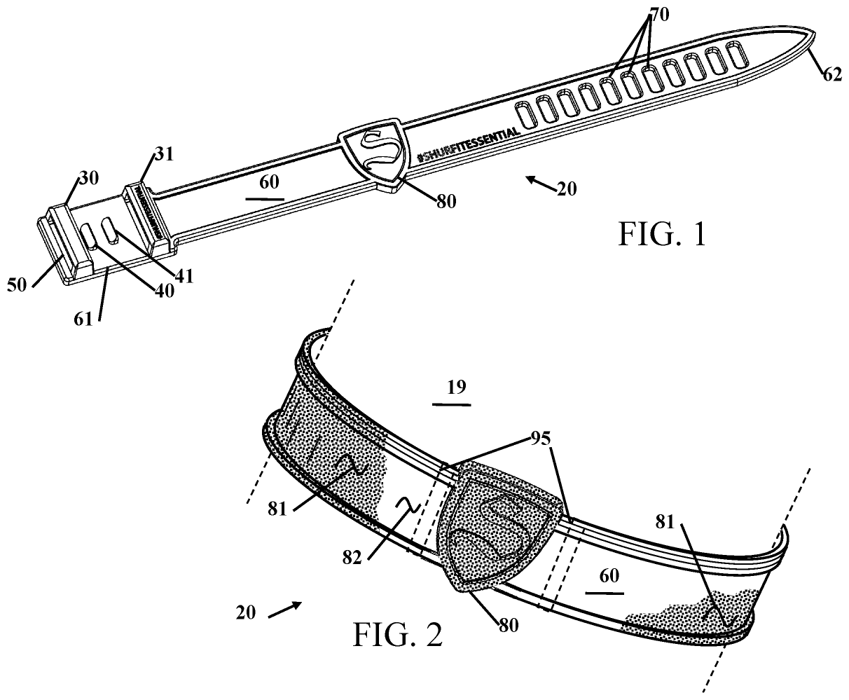

FIG. 1 shows a perspective view of a COVID thermal wristband 20. The COVID thermal wristband 20 is preferably molded with embedded and mixed liquid crystals that will be discussed in more detail with other figures and graphs in this document. Liquid crystals are used in precision applications, as their responses can be engineered to accurate temperatures, but their color range is limited by their principle of operation. In this embodiment the COVID thermal wristband 20 is configured as a wristband. While this embodiment is a single band with a connecting mechanism at both free ends, it is contemplated that the COVID thermal wristband 20 can be configured to secure to a watch or other jewelry, where the watch or jewelry replaced the emblem 80. In this figure, the strap front body 60 extends to the clasp end at one free end and to a strap tail 62 at the other free end.

The strap tail 62 is shown with a plurality of strap holes 70. The strap holes 70 are configured and spaced to connect with the front tab 40 and the rear tab 41 of the buckle. The strap is retained on the front tab 40 and the rear tab 41 within a bridge opening 50 where the strap tail 62 is configured to pass through the front bridge 30 and the rear bridge 31. While two securing tabs are shown and described it is contemplated that a single tab, 40 or 41 could be used or more than two tabs could be used to secure the strap hole(s) 70. The COVID thermal wristband 20 is intended to be a universal length, and after it is installed around a wrist (or ankle) the extra length of the strap tail 62 can be trimmed with scissors or the like.

Thermochromism is the property of a substance to change color due to temperature. Thermochromism is one of several types of chromism where chromism is a process that induces a change, often reversible, in the colors of compounds. This change is dependent on selective reflection of certain wavelengths by the crystallic structure of the material, as it changes between the low-temperature crystallic phase, through anisotropic chiral or twisted nematic phase, to the high-temperature isotropic liquid phase. Low-temperature state will reflect red-orange.

FIG. 2 shows a detailed view of the COVID thermal wristband 20 on a wrist or forearm 19 of a person. The area(s) of the wristband 20 that contacts the skin surface with an elevated temperature is show with the color change 81. Some areas of the strap front body 60 show the default (cold) color 82 change because they may not be in contact with the elevated skin temperature the back surface of the emblem 80 can be elevated from the body of the strap to create contact with the arm, wrist or forearm of a user to show a color change in the emblem 80. This figure also shows that the wristband can be configured as a watchband with clasps 95 that allow for the emblem to be replaced with a watch or other jewelry.

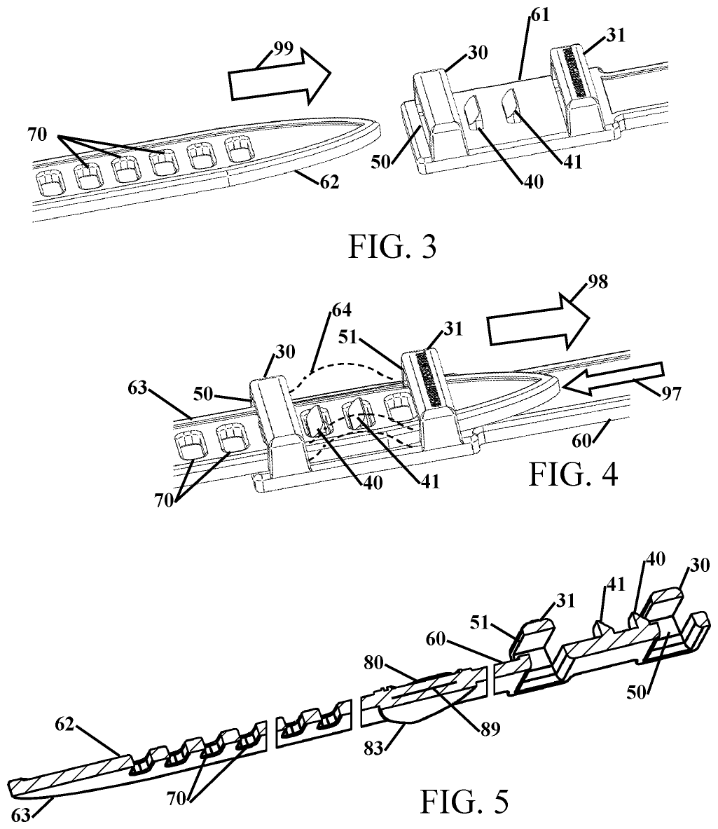

FIG. 3 shows the securing mechanism on the COVID thermal wristband with the mechanism displaced and FIG. 4 shows the securing mechanism on the COVID thermal wristband with the mechanism secured. The ends of the wristband are shown with a ratcheting strap where a user can simply install the band at a loose setting and pull on the loose end to tighten the strap around the wrist of the user. In an ideal size of the wristband the entire interior area of the wristband is in conductive contact with the skin of the wearer. The ratcheting is with one or more one-way teeth having a front tab 40 and a rear tab 41 with one side of the tabs are angled as teeth that are configured to engage and lock into one or more of the strap holes 70.

For installation the free end of the strap tail 62 is installed 99 into the front bridge opening 50 through the front bridge 30, over the tabs 40 and 41 on the strap buckle base 61 and then through the rear bridge opening 51 of the rear bridge 31. After the strap tail 62 is inserted through the rear bridge 31, a user can pull 98 on the strap tail 62 to “ratchet” additional strap holes 70 over the tabs 40 and 41 to shorten the length of the strap to tighten the strap on the arm or wrist of the user. Excessive length of the strap can be trimmed with scissors or the like. To remove the wristband the strap tail 62 can be pushed into the rear bridge 31 to slack 97 the loop 64 and remove the strap tail 62 from retention in the bridges. The wristband can be loosened (or tightened) by creating a “loop” 64 in the band over the one-way teeth or tabs 40 and 41.

FIG. 5 shows a cross-section view of the COVID thermal wristband through the vertical length of the wristband. Some portions of the wristband have been reduced to show the length of the wristband with minimal shrinking of the cross-section scale. Starting at the left side of the view the strap tail 63 has the strap front body 62 with a plurality of strap holes 70 that allows the inside diameter of the wristband to be adjusted for a proper fit on a person. The wristband material is a flexible elastomeric that allows the wristband to stretch and move with the user to maintain conductive contact with the person without reducing blood circulation.

One side of the wristband shows the emblem 80, and on the underside of the emblem is an elevated or proud 83 area that increases conductive contact with this particular portion of the body. An optional radio-frequency Identification (RFID) chip 89 is shown inserted within this area of the wristband. The RFID device does not require internal power for communication and can be used as identification to identify a wearer or to tract movement of the user for contact tracing by logging movement of the RFID tag. The RFID tag can provide anonymous tracking of a person until a band is identified as having an elevated temperature. The tracking database can then be queried to identify others that have been in the same general area. The database can identify a number, user ID, student ID or the name of people.

On the other end of the emblem 80 is the strap front body 60 that has a rear bridge 31 with a rear bridge opening 51. After the rear bridge are two tabs with one-way locking ramps, that are identified as the rear tab 41 and the front tab 40. There function has been previously discussed herein. The end to the wristband terminates with the rear opening 50 having the front bride 30 to retain the tail body 63 with the strap tail 62.

In a preferred embodiment the elastomeric material of the wristband in injection mold compatible material that is a combination of thermochromic pigment with a typical common universal copolymer. The color of the universal copolymer is selected as a gray color, but other colors other than black are contemplated. The color of the universal copolymer becomes less as the temperature increases until color of the universal copolymer becomes completely colorless or white. When the temperature of the color of the universal copolymer decreases the color becomes stronger returning to its original start color. This type of thermochromic plastic is referred to as a reversible plastic since it will repeat the noted color change an unlimited number of times. In the preferred embodiment the cold color is gray while the warm color is orange. One preferred ratio is 6% thermochromic pigment by weight to 80% universal copolymer by weight, but a ratio of 2%-30% thermochromic pigment is contemplated with the balance being universal copolymer depending upon the desired effect.

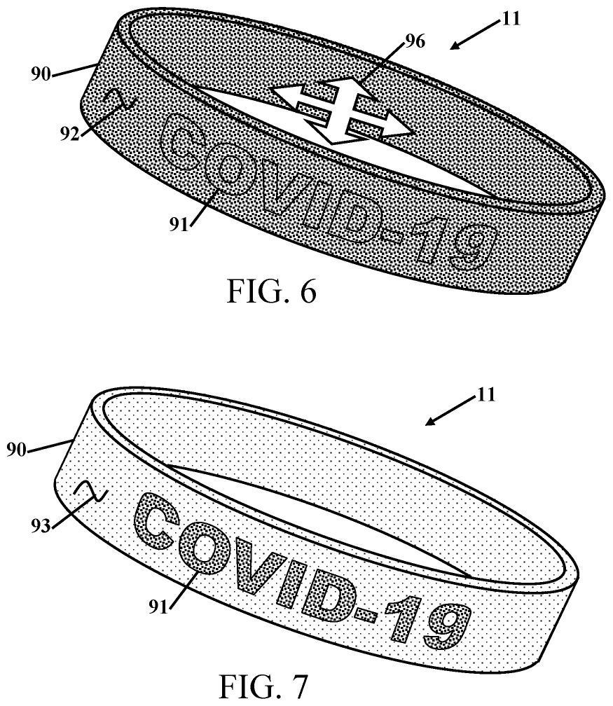

FIG. 6 shows a perspective view of an alternate embodiment of the COVID thermal wristband and FIG. 7 shows another view of the COVID thermal wristband showing an elevated temperature. In this embodiment the universal copolymer is silicon based to allow the alternate embodiment wristband 11 to stretch 96 in multiple directions to get over the hand of a person and onto the wrist or forearm of a user. The wristband has a ring shape. In this embodiment of the wristband 90 an area of indicia 91 is printed onto the wristband 90 or in a recess of the wristband 90. The color of the indicia 91 matches the cold color of the wristband, the default (cold) temperature or gray. While the temperature of the user remains cool, not sick, at or below 98.6 degrees the indicia is essentially invisible. When the user's body (and arm) temperature exceeds the thermal color change, the wristband 90 will change to the warm color 93 or orange temperature, but the color of the indicia (91) will remain at the default color or gray as shown in FIG. 7. While a particular color change of the band has been disclosed, a similar indication can take place if the indicia was printed in a thermochromic pigment whereby the color of the indicia 91 will change, but the remainder of the wristband will not change color.

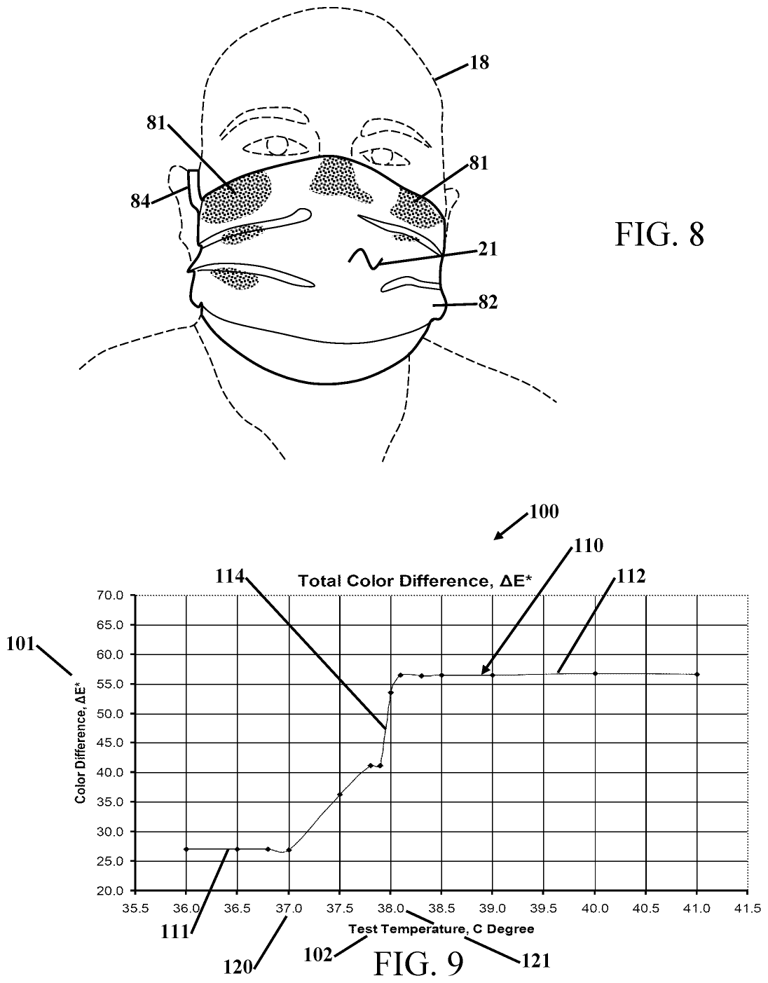

FIG. 8 shows the thermal material on a facemask 21. This figure shows the head 18 of a user with the facemask 21 secured over the ears with ear strap(s) 84 and around the mouth and nose of the person. The internal core temperature of the user is essentially the same as the temperature of air that they exhale. The facemask can be constructed from a single layer of material of multiple layers of material. In this figure the areas of the facemask 21 that are in conductive contact with the skin of the user shows a color change for the elevated temperature with some areas of the facemask 21 that are cooled by ambient air remain the default (cool) color 82. While this figure shows some or all of the material of the facemask being capable of showing an elevated temperature, it should be understood that specific areas of the mask can have thermochromic material or coating or the facemask can be printed with thermochromic indicia to show a word, slogan or other indicator. The thermochromic pigment is suspended in a carrier base that bonds the thermochromic pigment to the material of the facemask.

FIG. 9 shows a graph of the color change difference. In the graph the vertical axis is the color difference 101 and the horizontal axis is the test temperature 102. This color change graph 100 shows the plot 110 characteristics of general pigment color change where color change is based on resin and pigment ratio % adding less pigment % and more base resin is key to desired color spectrum. In this graph the ratio of pigment is 6% and 94% resin. This combination gives products response activation at body temperature of 98 degrees with a steady color change above switching to complete color change at 100.4 and above. The color change takes 10 to 30 seconds.

A significant change occurs at the lower inflection point 120 37 degrees C.-37.5 degrees C. where the color becomes significantly lighter with a more abrupt change—occurs at the upper inflection point—orange color 121 38 degrees C. color. The color change 112 after 38 Degrees C. is substantially stable and the change is marginal. The cold color 111 is temperatures below 37 degrees C. and the warm color 114 is temperatures above 38.0 degrees C. As a reference 37 degrees C. is 98.6 degrees F., while 38 degrees C. is a temperature of 100.4 degrees F.

Thus, specific embodiments of a COVID thermal wristband have been disclosed. It should be apparent, however, to those skilled in the art that many more modifications besides those described are possible without departing from the inventive concepts herein. The inventive subject matter, therefore, is not to be restricted except in the spirit of the appended claims.

1: A COVID thermal wristband comprising:
a wristband with a first end with a plurality of strap holes;
a second end of said wristband includes a clasp with two one-way angled locking ramps and that one is one side of another one-way angled locking ramp tab that creates a one-way locking tooth;
said wristband being configured to provide conductive heat transfer contact with a skin of a user through said wristband;
said wristband being a homogeneous material with a thermochromic pigment ratio of 2% to 30% thermochromic pigment by weight to universal copolymer by weight;
said thermochromic pigment changes color when exposed to a skin at a temperature of between 37 and 38 degrees C., and
whereby at least some of said wristband changes color when exposed to said skin at said skin temperatures above 38 degrees C.

2: The COVID thermal wristband according to claim 1, wherein there are at least two one-way angled locking ramp tabs.

3: The COVID thermal wristband according to claim 2, wherein said clasp has at least two open bridges that are located on an outside of the at least two one-way angled locking ramp tabs.

4: The COVID thermal wristband according to claim 3, wherein said first end of said wristband is configured to pass through said at least two open bridges and engage at least two of said plurality of strap holes in said at least two one-way angled locking ramp tabs.

5: The COVID thermal wristband according to claim 4, wherein said at least two one-way angled locking ramp tabs are configured to allow said first end to be pulled through said at least two open bridges to tighten said wristband.

6: The COVID thermal wristband according to claim 1, further includes a radio frequency identification (RFID) device.

7: The COVID thermal wristband according to claim 1, further includes an emblem between said first end and said second end of said wristband and said emblem extends beyond a front surface and a rear surface of said wristband.

8: The COVID thermal wristband according to claim 1, wherein said color change is orange at said temperature above 38 degrees C. and a color other than orange at said temperature below 37 degrees C.

9: The COVID thermal wristband according to claim 1, further including an indicium that is printed on at least some of an outer surface of said wristband wherein said indicium does not change color at temperatures above 37 degrees C.

10: The COVID thermal wristband according to claim 1, further includes a securing mechanism for a watch or jewelry.

11-20. (canceled)
